(19) United States
(12) Patent Application Publication
(10) Pub. No.: US 2024/0352001 A1
(43) Pub. Date: Oct. 24, 2024

(54) METHOD FOR PREPARING INTERMEDIATE FOR SYNTHESIS OF XANTHINE OXIDASE INHIBITOR

(71) Applicant: LG CHEM, LTD., Seoul (KR)

(72) Inventors: In Ae RYU, Seoul (KR); Ju Young LEE, Cheongju-si (KR); Joo Yong YOON, Seoul (KR); Seok Ju LEE, Seoul (KR); Ah Byeol PARK, Seoul (KR); Ki Dae KIM, Seoul (KR); Hui Rak JEONG, Seoul (KR)

(73) Assignee: LG CHEM, LTD., Seoul (KR)

(21) Appl. No.: 18/557,786

(22) PCT Filed: Apr. 26, 2022
(86) PCT No.: PCT/KR2022/005927

§ 371 Date: Oct. 27, 2023

(30) Foreign Application Priority Data
Apr. 27, 2021 (KR) 10-2021-0054152

(51) Int. Cl. C07D 403/04

(52) U.S. Cl. CPC C07D 403/04

(57) ABSTRACT

The present invention relates to a novel method for preparing an intermediate of Chemical Formula 3, wherein the intermediate can be effectively used in the synthesis of a xanthine oxidase inhibitor.

### TECHNICAL FIELD

The present invention relates to a method for preparing a key intermediate for the synthesis of a xanthine oxidase inhibitor, more specifically to a new method for preparing an intermediate of Chemical Formula 3 at a high yield without a separate separation process:

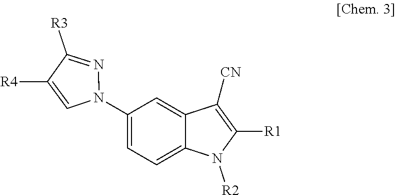

wherein
R1 is hydrogen, halogen, C₁-C₇ alkyl, C₁-C₇ alkoxy-C₁-C₇ alkyl or phenyl;
R2 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or

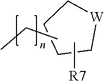

(wherein W represents O or S, R7 represents hydrogen or C₁-C₄ alkyl, and n is an integer 0 to 3);
R3 is hydrogen, halogen or C₁-C₇ alkyl; and
R4 is —C(O)OR8, wherein R8 is hydrogen, C₁-C₇ alkyl or C₃-C₇ cycloalkyl.

### BACKGROUND ART

Xanthine oxidase is known as an enzyme that converts hypoxanthine to xanthine and the formed xanthine to uric acid. Since uricase, which exists in most mammals, does not exist in humans and chimpanzees, a substance called uric acid is known to be the final product of purine metabolism (S. P. Bruce, Ann. Pharm., 2006, 40, 2187-2194). Uric acid contained in the blood at high concentrations causes various diseases, a representative example of which is gout.

As mentioned above, gout is a disease caused by high levels of uric acid in the body, and refers to a condition in which uric acid crystals accumulate in joint cartilage, ligaments, and surrounding tissues, causing severe inflammation and pain. Gout is a type of inflammatory joint disease, the incidence of which has been steadily increasing over the past 40 years (N. L. Edwards, Arthritis & Rheumatism, 2008, 58, 2587-2590).

Looking at the number of patients with gout in Western countries from the 1960s to the mid-1990s, there is a surprising increase of about 200% to 300%, and patients with gout are mainly men. Obesity, aging, decreased kidney function, high blood pressure, and the like are considered to be the causes of the rate of increase in patients with gout. The incidence of gout is about 1.4/1000 people, but also vary depending on the level of uric acid. In other words, the incidence of gout in patients having a uric acid level in the blood of 7.0 mg/dl or more is 0.5%, but the incidence of gout in patients having a uric acid level in the blood of 9.0 mg/dl or more is 5.5% (G. Nuki, Medicine, 2006, 34, 417-423). Considering the incidence, it can be seen that uric acid concentration in the blood is an important factor causing gout. Additionally, dietary habits, alcohol, lipids, obesity, and the like may also act as important factors causing gout. These days, studies on the correlation between uric acid and heart failure, high blood pressure, diabetes, kidney disease, and cardiovascular disease are being actively carried out by a number of researchers, and the importance of uric acid management is increasing (D. I. Feig et al., N. Eng. J. Med, 2008, 23, 1811-1821). In addition, allopurinol, a xanthine oxidase inhibitor, is known to be effective in ulcerative colitis (Aliment. Pharmacol. Ther. 2000, 14, 1159-1162; WO 2007/043457).

Until febuxostat was approved as an arthrifuge in the United States in 2009 (Brain Tomlinson, Current opin. invest. drugs, 2005, 6, 1168-1178), allopurinol was the only drug used to treat gout for the past 40 years. Allopurinol is known as a non-specific inhibitor of various enzymes involved in purine and pyrimidine metabolism, and has a Ki of 700 nM against xanthine oxidase (Y. Takano et al., Life Sciences, 2005, 76, 1835-1847). Allopurinol is directly oxidized by xanthine oxidase and converted to oxypurinol, and this metabolite is known to act as a greatly strong inhibitor of xanthine oxidase.

However, allopurinol is known to cause gastrointestinal side effects and skin rashes, and to have poor compliance when taken for a long period of time. In particular, among patients taking allopurinol, it has been reported that an unpredictable fatal side effect of Stevens-Johnson syndrome occurs at a low ratio (Felix Arellano et al, Ann. Pharm., 1993, 27, 337-43). This side effect is known to be a serious side effect that causes cell necrosis in the skin and mucous membranes of the mouth, leading to death in about 25% of cases if not treated appropriately.

Accordingly, various studies have been conducted to develop new xanthine oxidase inhibitors, and Korean Patent Publication No. 10-2011-0037883 discloses a novel compound of the following Chemical Formula 1, which is effective as a xanthine oxidase inhibitor:

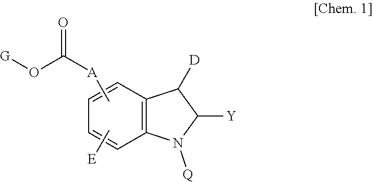

in Chemical Formula 1,
A is selected from the following substituents A-i, A-ii, A-iii, A-iv, A-v, A-vi, A-vii and A-viii,

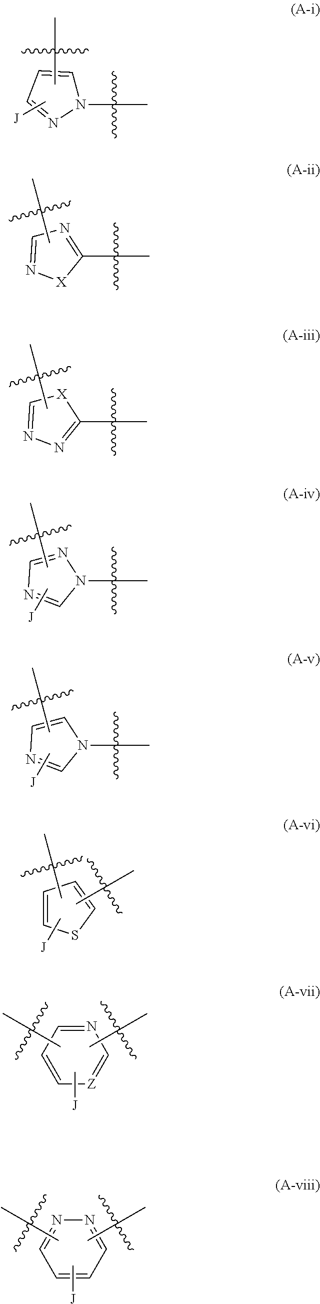

wherein
J represents hydrogen, halogen, or C₁-C₆-alkyl unsubstituted or substituted with halogen,
X is O or S, and
Z is C or N,
E represents hydrogen, halogen, cyano, nitro, substituted or unsubstituted C₁-C₆-alkyl, or substituted or unsubstituted C₁-C₆-alkoxy,
D represents hydrogen, halogen, cyano, nitro, C₁-C₆-alkyl unsubstituted or substituted with halogen, —CHO, or —CH═N—OH,
Q is selected from the following substituents Q-i, Q-ii, and Q-iii-1 to Q-iii-9 (Q-i) hydrogen;
(Q-ii) substituted or unsubstituted linear, branched or cyclic, saturated or unsaturated alkyl;

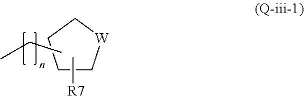

(wherein W represents O or S, R7 represents hydrogen or substituted or unsubstituted lower alkyl, and n is an integer 0 to 3);

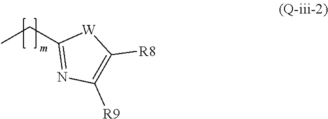

(wherein W represents O or S, R8 and R9 each independently represent hydrogen or lower alkyl, and m is an integer 1 to 3);

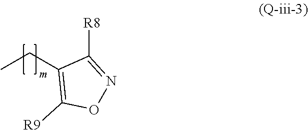

(wherein R8 and R9 each independently represent hydrogen or lower alkyl, and m is an integer 1 to 3);

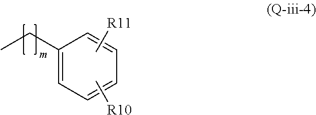

(wherein, R10 and R11 each independently represent hydrogen, halogen, lower alkoxy, or lower alkyl, and m is an integer 1 to 3);

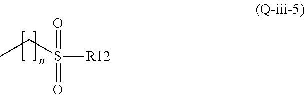

(wherein R12 represents substituted or unsubstituted lower alkyl or aromatic, and n is an integer 0 to 3);

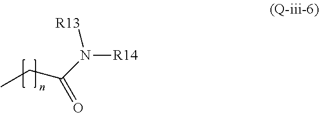

(wherein, R13 and R14 each independently represent substituted or unsubstituted lower alkyl, or may form a 3- to 7-membered heterocycle containing N, and n is an integer 0 to 3);

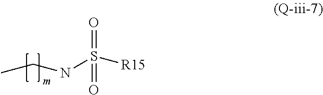

(wherein R15 represents substituted or unsubstituted lower alkyl, and m is an integer 1 to 3),

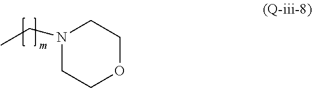

(wherein m is an integer 1 to 3); and

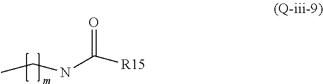

(wherein R15 represents substituted or unsubstituted lower alkyl, and m is an integer 1 to 3),
Y represents hydrogen, halogen, substituted or unsubstituted linear, branched or cyclic saturated or unsaturated alkyl, substituted or unsubstituted C₁-C₆-alkoxy, substituted or unsubstituted aromatic, or heteroaromatic, and
G represents hydrogen or substituted or unsubstituted linear, branched or cyclic, saturated or unsaturated alkyl.

In a specific example of the document, the preparation of 1-(3-cyano-1-isopropyl-indol-5-yl) pyrazole-4-carboxylic acid according to the following Scheme 1 is disclosed.

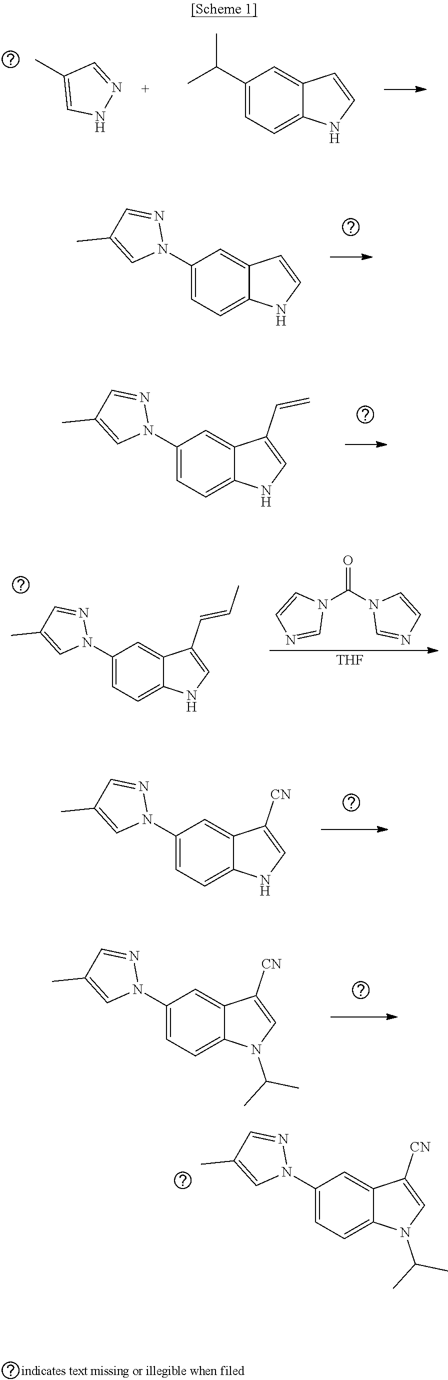

In Scheme 1, a synthesis method (86% yield) through a three-step process is disclosed, in which 1-(1H-indol-5-yl) pyrazole-4-carboxylic acid ethyl ester is added to the reaction solution of oxalyl chloride and N,N-dimethylformamide, then the reaction is conducted, the organic layer is dried over anhydrous magnesium sulfate and concentrated under reduced pressure to prepare 1-(3-cyano-1H-indol-5-yl) pyrazole-4-carboxylic acid ethyl ester, then hydroxyammonium chloride is added thereto, the mixture is heated and stirred under reflux, when the reaction is completed, the reaction mixture is concentrated under reduced pressure and filtered through silica gel to prepare 1-[3-[(E,Z)-hydroxyiminomethyl]-1H-indol-5-yl]pyrazole-4-carboxylic acid ethyl ester, di(imidazol-1-yl) methanethione is added thereto, the reaction is conducted at room temperature while stirring is performed, the reaction mixture is concentrated under reduced pressure, and the produced solid compound is separated by column chromatography to prepare 1-(3-cyano-1H-indol-5-yl) pyrazole-4-carboxylic acid ethyl ester.

However, the method includes several synthesis steps, and has a problem of not being preferable for mass synthesis at high yields.

### SUMMARY OF INVENTION

### Technical Problem

Accordingly, the technical object of the present invention is to provide a method suitable for more efficient mass production of a compound of Chemical Formula 3, which is a key intermediate in the synthesis of an excellent xanthine oxidase inhibitor.

### Solution to Problem

In order to achieve the object, the present invention provides a method for preparing a compound of the following Chemical Formula 3 from a compound of the following Chemical Formula 2 through one step in the same container in the same solvent system without separation of intermediates:

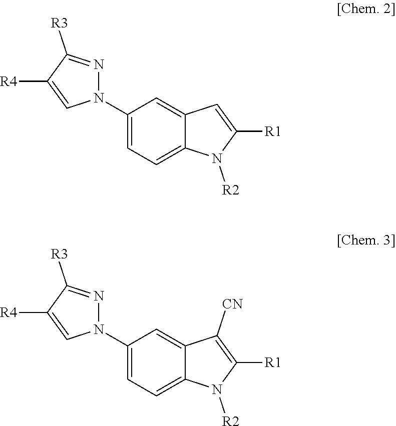

wherein,
R1 is hydrogen, halogen, C₁-C₇ alkyl, C₁-C₇ alkoxy-C₁-C₇ alkyl or phenyl;
R2 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or

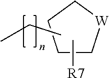

(wherein W represents O or S, R7 represents hydrogen or C₁-C₄ alkyl, and n is an integer 0 to 3);
R3 is hydrogen, halogen or C₁-C₇ alkyl; and
R4 is —C(O)OR8, wherein R8 is hydrogen, C₁-C₇ alkyl or C₃-C₇ cycloalkyl.

Hereinafter, the present invention will be described in detail.

In the present invention, a compound of Chemical Formula 3 is prepared by reacting a compound of Chemical Formula 2 with chlorosulfonyl isocyanate and triethylamine in an organic solvent.

In an embodiment according to the present invention, as the organic solvent in the method, for example, one or more selected from ethyl acetate, dimethylformamide (DMF), acetonitrile, acetone, tetrahydrofuran (THF), or dichloromethane may be used.

In an embodiment according to the present invention, dimethylformamide (DMF) may be used instead of triethylamine (NEt₃) in the method.

In the present invention, the compound of Chemical Formula 3 can be prepared through one-pot reactions by forming —CONHSO₂Cl as an intermediate using the compound of Chemical Formula 2 and chlorosulfonyl isocyanate, and introducing a cyano group using triethylamine or dimethylformamide without additional separation or purification processes.

### Advantageous Effects of Invention

In the preparation method of the present invention, a compound of Chemical Formula 3 can be mass-produced from a compound of Chemical Formula 2 through one step in the same container in the same solvent system without separation of intermediates at a high yield.

### DESCRIPTION OF EMBODIMENTS

Hereinafter, the present invention will be described in more detail with reference to Example. However, the following Example is merely illustrative to aid understanding of the present invention, and the scope of the present invention is not limited thereto.

### Example: Synthesis of 1-(3-cyano-1H-indol-5-yl) pyrazole-4-carboxylic acid ethyl ester

1-(1H-indol-5-yl) pyrazole-4-carboxylic acid ethyl ester (20 g, 78.3 mmol) was dissolved in 80 ml of ethyl acetate (EtOAc), and then chlorosulfonyl isocyanate (12.20 g, 86.2 mmol) diluted with 20 ml of ethyl acetate was slowly added at 0° C. to 5° C. Stirring was performed for 1 hour, then triethylamine (Et₃N, 8.72 g, 86.2 mmol) diluted with 20 ml of ethyl acetate was slowly added at 0° C. to 5° C., and stirring was performed at room temperature for 1 hour. Ethyl acetate was additionally added to the reaction mixture, washing with 5% NaHCO₃ aqueous solution was performed, and the organic layer was filtered through Na₂SO₄/Celite. The solvent was distilled off under reduced pressure, and crystallization with toluene was performed to obtain 21.7 g (98% yield) of the title compound.

¹H-NMR (CD₃OD) δ 8.74 (1H, s), 8.06 (2H, d), 8.03 (1H, d), 7.72 (1H, dd), 7.63 (1H, d), 4.32 (2H, q), 1.36 (3H, t)

1. A method for preparing a compound of Chemical Formula 3, the method comprising adding chlorosulfonyl isocyanate and triethylamine to a compound of the following Chemical Formula 2 in an organic solvent and conducting one-pot reaction: 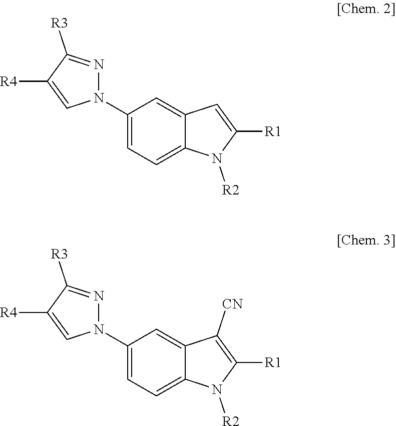
in the chemical formulas,
R1 is hydrogen, halogen, C₁-C₇ alkyl, C₁-C₇ alkoxy-C₁-C₇ alkyl or phenyl;
R2 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or 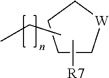
(wherein W represents O or S, R7 represents hydrogen or C₁-C₄ alkyl, and n is an integer 0 to 3);
R3 is hydrogen, halogen or C₁-C₇ alkyl; and
R4 is —C(O)OR8, wherein R8 is hydrogen, C₁-C₇ alkyl or C₃-C₇ cycloalkyl.

2. The preparation method according to claim 1, wherein the organic solvent is one or more selected from ethyl acetate, dimethylformamide, acetonitrile, acetone, tetrahydrofuran, or dichloromethane.

3. The preparation method according to claim 1, wherein dimethylformamide is used instead of triethylamine.
